(19) United States
(12) Patent Application Publication
(10) Pub. No.: US 2019/0357865 A1
(43) Pub. Date: Nov. 28, 2019

(54) USER INTERFACE FOR PORTABLE X-RAY DETECTOR

(71) Applicant: General Electric Company, Schenectady, NY (US)

(72) Inventors: Nicholas Ryan Konkle, Sussex, NY (US); Patrick Jeffrey Harrington, Milwaukee, WI (US); Catherine Noel Bailey, Waukesha, WI (US); Daniel Robert Lochner, West Allis, WI (US); Kevin Edward Kinsey, Waukesha, WI (US); William Andrew Hennessy, Troy, NY (US); Joseph Kulak, Waukesha, WI (US); Douglas Albagli, Troy, NY (US)

(21) Appl. No.: 15/989,425

(22) Filed: May 25, 2018

(51) Int. Cl. A61B 6/00

(52) U.S. Cl. CPC A61B 6/46; A61B 6/4405; A61B 6/4233

(57) ABSTRACT

A digital X-ray detector is provided. The detector includes a detector array configured to generate image data based on incident X-ray radiation. The detector also includes a housing in which the detector array is disposed. The detector further includes an indicator disposed adjacent a corner of the housing, wherein the indicator includes at least one light source and is configured to provide a user perceptible signal indicating a status of the digital X-ray detector.

### BACKGROUND

The subject matter disclosed herein relates to X-ray imaging systems and more particularly to a user interface of a portable X-ray detector.

A number of radiological imaging systems of various designs are known and are presently in use. Such systems generally are based upon generation of X-rays that are directed toward a subject of interest. The X-rays traverse the subject and impact a film or a digital detector. In medical diagnostic contexts, for example, such systems may be used to visualize internal tissues and diagnose patient ailments. In other contexts, parts, baggage, parcels, and other subjects may be imaged to assess their contents and for other purposes.

Increasingly, such X-ray systems use digital circuitry, such as solid-state detectors, for detecting the X-rays, which are attenuated, scattered or absorbed by the intervening structures of the subject. Solid-state detectors may generate electrical signals indicative of the intensities of received X-rays. These signals, in turn, may be acquired and processed to reconstruct images of the subject of interest.

As digital X-ray imaging systems have become increasingly widespread, digital X-ray detectors have become more portable for even greater versatility. These digital X-ray detectors may include user interfaces that provide numerous information about the detectors (e.g., temperature, charge status, Wi-Fi status, errors, etc.) to the user. The amount of information about the detector may be overwhelming to the user. In addition, the information about the detector may not be used by the user. Another issue with the digital X-ray detectors is that it might be difficult to determine the active area of a detector (e.g., during positioning of an X-ray source) when the detector is disposed underneath a patient.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of possible forms of the subject matter. Indeed, the subject matter may encompass a variety of forms that may be similar to or different from the embodiments set forth below.

In accordance with a first embodiment, a digital X-ray detector is provided. The detector includes a detector array configured to generate image data based on incident X-ray radiation. The detector also includes a housing in which the detector array is disposed. The detector further includes an indicator disposed adjacent a corner of the housing, wherein the indicator includes at least one light source and is configured to provide a user perceptible signal indicating a status of the digital X-ray detector.

In accordance with a second embodiment, a digital X-ray detector is provided. The detector includes a detector array configured to generate image data based on incident X-ray radiation. The detector also includes a housing in which the detector array is disposed. The housing includes multiple corners. The detector further includes multiple indicators. Each indicator of the multiple indicators is located at a respective corner of the plurality of corners. Each indicator of the multiple indicators includes at least one light source. Each indicator of the multiple indicators includes multiple indicators that together are configured to provide a single user perceptible signal indicating a single status of the digital X-ray detector.

In accordance with a third embodiment, a method is provided. The method includes determining a single status of a digital X-ray detector. The method includes providing a user perceptible signal indicating the single status of the digital X-ray detector via an indicator disposed adjacent a corner of a housing of the digital X-ray detector, wherein the indicator includes at least one light source.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical overview of a digital X-ray imaging system in which the present technique may be utilized;

FIG. 2 is a diagrammatical overview of the detector of FIG. 1;

FIG. 3 is a perspective view of an embodiment of a detector having indicators;

FIG. 4 is a partial top view of an embodiment of a detector corner having an indicator (e.g., L-shaped indicator);

FIG. 5 is a partial top view of an embodiment of a detector corner having an indicator (e.g., square-shaped indicator);

FIG. 6 is a partial top view of an embodiment of a detector corner having an indicator (e.g. spaced arrangement);

FIG. 7 is a partial top view of an embodiment of a detector corner having an indicator;

FIG. 8 is a partial cross-sectional view of the detector corner of FIG. 7, taken within line 8-8;

FIG. 9 is a side perspective view of a portion of an indicator; and

FIG. 10 is a bottom perspective view of a portion of the indicator of FIG. 9.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

The following embodiments describe a portable digital X-ray detector that includes one or more indicators located adjacent a respective one or more corners of a housing of the detector. The indicators provide a user perceptible signal (e.g., via light source) of a status of the detector while also identifying or marking an active area of the detector for acquiring image data. In certain embodiments, the one or more indicators provide a single signal indicating a single status of the indicator (e.g., whether or not the detector is ready for acquiring image data). In certain embodiments, each indicator indicates the active area in both a first direction and a second direction (e.g., crosswise to the first direction). The one or more indicators may make it easier to identify the active area of the detector when a patient is disposed on the detector. In addition, the one or more indicators may provide a user interface to provide a simple indication of the status of the detector without providing too much information that typically would be ignored by the user.

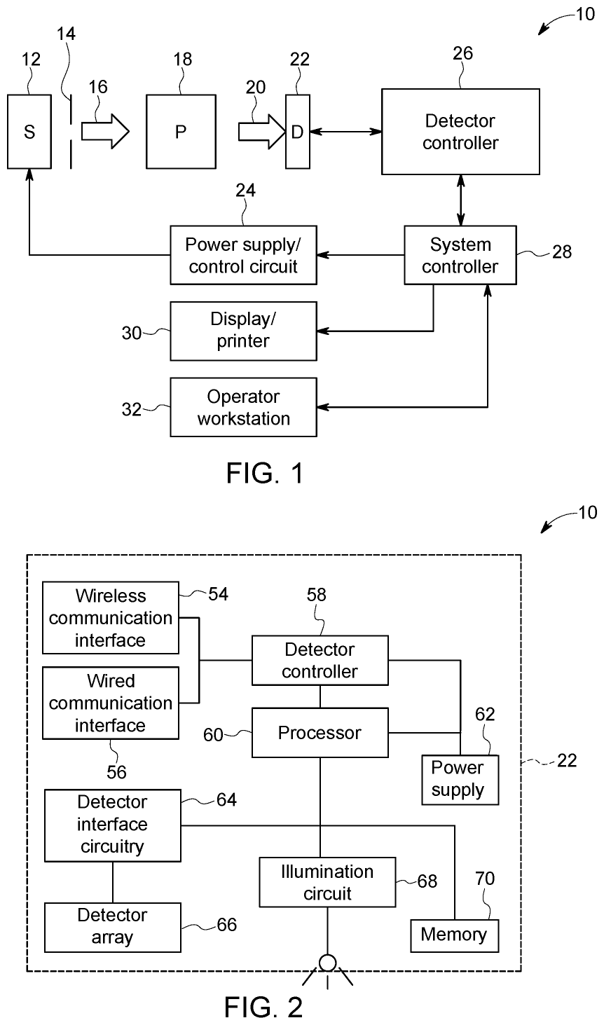

Turning now to the drawings, FIG. 1 illustrates diagrammatically an imaging system 10 for acquiring and processing discrete pixel image data. In the illustrated embodiment, system 10 is a digital X-ray system designed both to acquire original image data and to process the image data for display in accordance with the present technique. The imaging system 10 may be a stationary system disposed in a fixed X-ray imaging room or a mobile X-ray system. In the embodiment illustrated in FIG. 1, imaging system 10 includes a source of X-ray radiation 12 positioned adjacent to a collimator 14. Collimator 14 permits a stream of radiation 16 to pass into a region in which a subject, such as a human patient 18 is positioned. A portion of the radiation 20 passes through or around the subject and impacts a digital X-ray detector, represented generally at reference numeral 22. The detector 22 may be portable or permanently mounted to the system 10. In certain embodiments, the detector 22 may convert the X-ray photons incident on its surface to lower energy photons, and subsequently to electric signals, which are acquired and processed to reconstruct an image of the features within the subject. In other embodiments, such as in a direct conversion implementation, the incident radiation itself may be measured without an intermediary conversion process.

Source 12 is controlled by a power supply/control circuit 24 which furnishes both power and control signals for examination sequences. Moreover, detector 22 is coupled to a detector controller 26 which commands acquisition of the signals generated in the detector 22. Detector controller 26 may also execute various signal processing and filtration functions, such as for initial adjustment of dynamic ranges, interleaving of digital image data, and so forth. Both power supply/control circuit 24 and detector controller 26 are responsive to signals from a system controller 28. In general, system controller 28 commands operation of the imaging system to execute examination protocols and to process acquired image data. In the present context, system controller 28 also includes signal processing circuitry, typically based upon a general purpose or application-specific digital computer; and associated manufactures, such as optical memory devices, magnetic memory devices, or solid-state memory devices, for storing programs and routines executed by a processor of the computer to carry out various functionalities (e.g., gain calibration and gain correction), as well as for storing configuration parameters and image data; interface protocols; and so forth. In one embodiment, a general or special purpose computer system may be provided with hardware, circuitry, firmware, and/or software for performing the functions attributed to one or more of the power supply/control circuit 24, the detector controller 26, and/or the system controller 28 as discussed herein.

In the embodiment illustrated in FIG. 1, system controller 28 is linked to at least one output device, such as a display or printer as indicated at reference numeral 30. The output device may include standard or special purpose computer monitors and associated processing circuitry. One or more operator workstations 32 may be further linked in the system for outputting system parameters, requesting examinations, viewing images, and so forth. In general, displays, printers, workstations, and similar devices supplied within the system may be local to the data acquisition components, or may be remote from these components, such as elsewhere within an institution or hospital, or in an entirely different location, linked to the image acquisition system via one or more configurable networks, such as the Internet, virtual private networks, and so forth.

FIG. 2 illustrates diagrammatically the detector 22 of FIG. 1. The detector 22 includes a wireless communication interface 54 for wireless communication with the imaging system 10. In certain embodiments, the detector 22 may also include a wired communication interface 56, for communicating with the detector when it is tethered to a component of the imaging system 10. The detector 22 may also be in communication with a server. It is noted that the wireless communication interface 54 may utilize any suitable wireless communication protocol, such as an ultra-wideband (UWB) communication standard, a Bluetooth communication standard, or any 802.11 communication standard. Moreover, detector 22 is coupled to a detector controller 58 which coordinates the control of the various detector functions. For example, detector controller 58 may execute various signal processing and filtration functions, such as for initial adjustment of dynamic ranges, interleaving of digital image data, and so forth. The detector controller 58 is responsive to signals from the system controller 28. The detector controller 58 is linked to a processor 60. The processor 60, the detector controller 58, and all of the circuitry receive power from a power supply 62. The power supply 62 may include a battery. Alternatively, the detector 14, including the power supply 62, may receive power from a power supply when tethered to another component of the imaging system 10.

Also, the processor 60 is linked to detector interface circuitry 64. The detector 22 converts X-ray photons received on its surface to lower energy photons. The detector 22 includes a detector array 66 that includes an array of photodetectors to convert the light photons to electrical signals. Alternatively, the detector 22 may convert the X-ray photons directly to electrical signals. These electrical signals are converted to digital values by the detector interface circuitry 64 which provides the values to the processor 60 to be converted to imaging data and sent to the imaging system 10 (e.g., system controller 28, operator workstation 32, etc.) to reconstruct an image of the features within a subject. Alternatively, the imaging data may be sent from the detector 22 to a server to process the imaging data.

The processor 60 is also linked to an illumination circuit 68. The detector controller 58, in response to a signal received from the imaging system 10, may send a signal to the processor 60 to signal the illumination circuit 68 to illuminate one or more lights to indicate the active status (or where desired, any status) of the detector 22 in response to the signal. Further, the processor 60 is linked to a memory 70. The memory 70 may store various configuration parameters, calibration files, and detector identification data. As described in greater detail below, the illumination circuit 68 may be coupled to one or more indicators located at or adjacent one or more respective corners of the detector 22. In certain embodiments, one or more indicators may be located anywhere along a side extending between adjacent corners of the detector 22. Each indicator may include one or more light sources (e.g., light emitting diodes (LEDs)). In certain embodiments, the indicators may together provide a single user perceptible signal (i.e., light up together and in the same manner) to indicate a single status of the detector 22 (e.g., whether the detector 22 is ready for acquiring image data). The readiness of the detector 22 may take into account numerous factors (e.g., Wi-Fi status, battery status, presence of errors, etc.). Providing a single user perceptible signal related to the readiness of the detector 22 provides a simple, useful user interface for the user. In addition, the one or more indicators may be located on the detector 22 (e.g., in the corners) in a manner that indicates the active area of the detector to help in positioning the X-ray source.

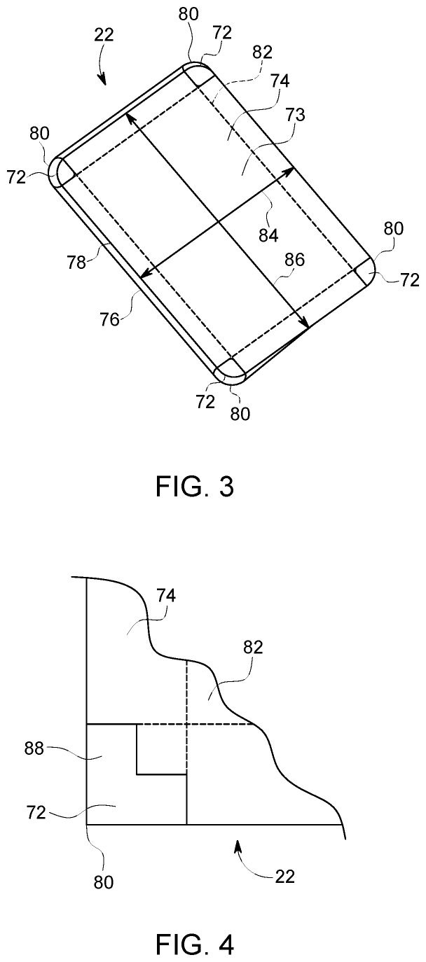

FIG. 3 is a perspective view of an embodiment of the detector 22 having indicators 72. As depicted, the detector 22 includes a cover 73 that encloses the components of the detector 22 (e.g., detector array 66, illumination circuit 68, etc.). The cover 73 may be made of metal, metal alloy, plastic, a composite material, or a combination thereof. For example, the cover 73 may be made of a lightweight, durable composite material such as carbon fiber. The detector 22 includes a top surface 74, a bottom surface 76 disposed opposite the top surface 74, and side surfaces 78 extending between the top and bottom surfaces 74, 76. The detector 22 includes multiple corners 80 (e.g., four corners 80). As depicted, indicators 72 are located at or adjacent each corner 80 (e.g., four indicators 72). In certain embodiments, only some corners 80 may include a respective indicator 72 (e.g., 1 corner, 2 corners, 3 corners, etc.). In certain embodiments, indicators 72 be disposed along the detector 22 at any point between adjacent corners 80. As depicted, each indicator 72 extends from the top surface 74 across the side surface 78 to the bottom surface 76. In certain embodiments, each indicator 72 may only be present on a single surface 74, 76, 78 or extend over some but not all of the surfaces 74, 76, 78.

Each indicator 72 may include one or more light sources (e.g., LEDs) disposed within the detector 22. In certain embodiments, each indicator 72 may include a translucent or transparent material (e.g., light plastic material) disposed over the light sources that functions as a light guide. In some embodiments, each indicator 72 may include a strong material robust to cracking (e.g., metal such as stainless steel, hard plastic, etc.) configured to absorb a mechanical shock to the corner 80 to keep the translucent or transparent material from breaking.

In certain embodiments, the indicators 72 act as a single user interface communicating a single user perceptible signal representing a single status of the detector 22 (e.g., whether the detector 22 is ready for acquiring image data). For example, the indicators 72 may remain off and only turn on (e.g., light up) when the detector 22 is ready. Alternatively, the indicators 72 may light up as a first color to indicate the detector 22 is ready and light up as a different second color when the detector 22 is not ready (e.g., due to error, low battery, etc.). In some embodiments, the indicators 72 may flash. In some embodiments, the indicators 72 may be utilized separately. In other embodiments, the indicators 72 may be utilized to separately indicate multiple different types of information related to the detector 22 (e.g., Wi-Fi strength, battery level, errors due to temperature and/or mechanical shock, etc.).

Besides indicating the status of the detector 22, the indicators 72 also indicate an active area 82 (edges of the active area 82) of the detector 22 for acquiring the image data. For example, when lit up, each indicator 72 indicates the active area 82 in a first direction 84 (e.g., along a width of the detector 22) and in a second direction 86 (e.g., along a longitudinal length of the detector 22) crosswise (e.g., orthogonal or perpendicular) to the first direction 84. In certain embodiments, each indicator 72 include a first light source or first set of light sources to indicate the active area 82 in the first direction 84 and a second light source or second set of light sources to indicate the active area 82 in the second direction 86. The indicators 72 make it easier for the user to visualize the active area for positioning the X-ray source when the detector 22 is disposed underneath a patient. The utilization of the indicators 72 helps avoid missing any desired anatomy during imaging.

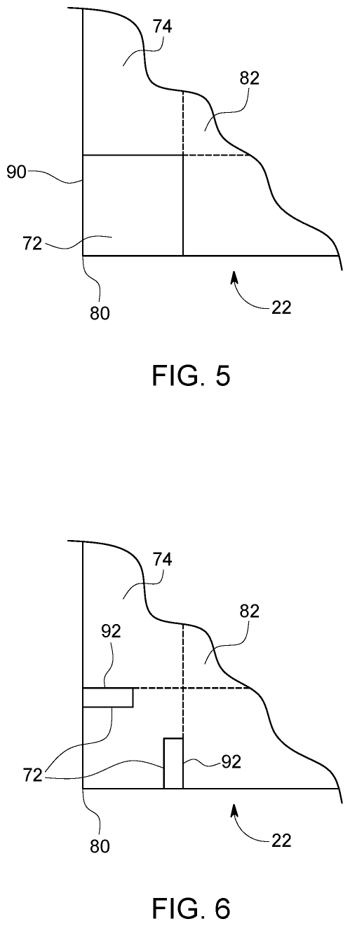

FIGS. 4-6 are different partial top views of detector corners 80 having different shaped indicators 72. FIG. 4 illustrates an L-shaped indicator 88. FIG. 5 illustrates a square-shaped indicator 90. FIG. 6 illustrates an indicator 92 with a spaced arrangement (e.g., light sources or sets of light sources flanking each side of the corner 80). As depicted in FIGS. 4-6, the outer edges of the indicators 72 mark or identify the active area 82 of the detector 22. Although depicted on the top surface 74 of the detector 22, the indicators 72 may extend from the top surface 74 across the side surface 78 to the bottom surface 76. In certain embodiments, the indicators 72 may only be present on a single surface 74, 76, 78 or extend over some but not all of the surfaces 74, 76, 78.

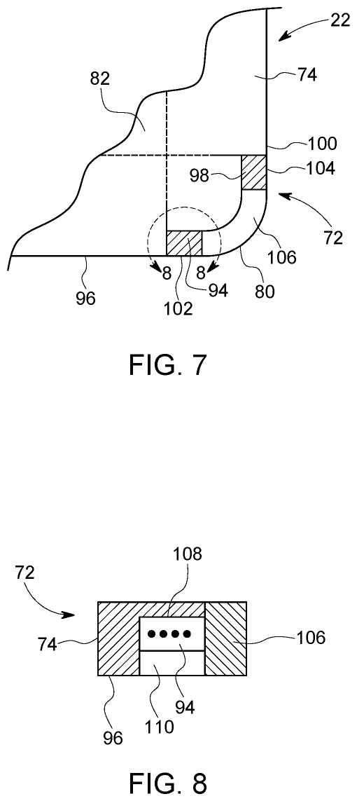

FIG. 7 is a partial top view of an embodiment of the detector corner 80 having the indicator 72 with a spaced arrangement (e.g., similar to FIG. 6). The indicator 72 includes a first light source or first set of light sources 94 disposed on a first side 96 of the corner 80 and a second light source or second set of light sources 98 disposed on a second side 100 of the corner 80. The number of light sources in a set of light sources may vary. Respective edges 102, 104 of the light sources 94, 98 indicate the active area 82 of the detector 22. The light sources 94, 98 flank a corner material 106 that extends around the corner 80. The corner material 106 may include a shock absorbing material (e.g., metal such as stainless steel, hard plastic, etc.) configured to absorb a mechanical shock to the corner 80 to keep components of the indicator 72 (e.g., a translucent or transparent material (see FIGS. 8-10)) from breaking. As depicted, the indicator 72 is flush with the sides 96, 98 of the detector 22. In certain embodiments, a portion of the indicator 72 may extend beyond the sides 96, 98.

As depicted in FIG. 8, the light source (or set of light sources) 94 is embedded within the detector 22. In particular, the light source 98 is disposed within a recess 108 between the cover 73 (e.g., carbon fiber material) of the detector 22 and the corner material 106. A translucent or transparent material 110 (e.g., light plastic material) is disposed over the light source 98. The material 110 is also fire retardant and biocompatible. The translucent material functions as a light guide for the light source 98. The light source 98 of the indicator 72 may be similarly arranged as light source 94.

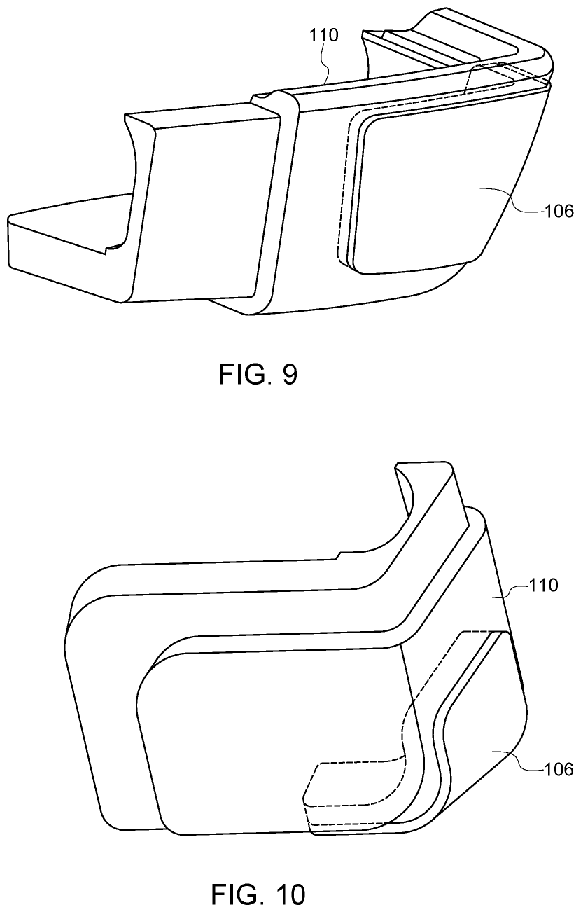

FIGS. 9 and 10 are different perspective views of a portion (e.g., translucent material 110 and corner material 106) of the indicator 72. As depicted in FIGS. 9 and 10, the translucent material 110 may form a single piece that couples to and extends around the corner 80 of the detector 22 (e.g., from side 96 to side 100 around the corner 80). The translucent material 110 is as described above. The translucent material 110 may fit over a portion of surfaces 74, 76, and/or 78 of the detector 22. As noted above, the light sources may be embedded in the detector 22. As depicted, the corner material 106 may extend along a portion of the translucent material 110, in particular, the portion of the translucent material 110 extending around the corner 80. The corner material 106 is configured to absorb a mechanical shock to the corner 80 to keep the translucent material 110 from breaking.

Technical effects of the disclosed embodiments include providing one or more indicators located adjacent a respective one or more corners of a housing of a detector. The indicators provide a user perceptible signal (e.g., via light source) of a status of the detector while also identifying or marking an active area of the detector for acquiring image data. In certain embodiments, the one or more indicators provide a single signal indicating a single status of the indicator (e.g., whether or not the detector is ready for acquiring image data). The one or more indicators may provide a user interface to provide a simple indication of the status of the detector without providing too much information that typically would be ignored by the user. In addition, the one or more indicators may make it easier to identify the active area of the detector when a patient is disposed on the detector.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A digital X-ray detector, comprising:
a detector array configured to generate image data based on incident X-ray radiation;
a housing in which the detector array is disposed; and
an indicator disposed adjacent a corner of the housing, wherein the indictor comprises at least one light source and is configured to provide a user perceptible signal indicating a status of the digital X-ray detector.

2. The digital X-ray detector of claim 1, wherein the indicator is configured to identify an active area of the digital X-ray detector for acquiring the image data.

3. The digital X-ray detector of claim 2, wherein the indicator is configured to indicate the active area in a first direction and a second direction crosswise to the first direction.

4. The digital X-ray detector of claim 3, wherein the indicator comprises a first light source that is configured to indicate the active area in the first direction and a second light source configured to indicate the active area in the second direction.

5. The digital X-ray detector of claim 1, wherein the indicator is configured to indicate a single status of the digital X-ray detector.

6. The digital X-ray detector of claim 5, wherein the single status comprises whether the digital X-ray detector is ready for acquiring the image data.

7. The digital X-ray detector of claim 1, wherein the indicator comprises a translucent light guide disposed over the at least one light source.

8. The digital X-ray detector of claim 7, wherein the indicator comprises a material resistant to breakage configured to distribute a mechanical shock to the corner to protect the translucent light guide from breaking.

9. The digital X-ray detector of claim 8, wherein the material resistant to breakage is disposed over a portion of the translucent light guide.

10. The digital X-ray detector of claim 8, wherein the indicator comprises a first light source and a first translucent light guide disposed over the first light source at a first location adjacent the corner, a second light source and a second translucent light guide disposed over the second translucent guide at a second location adjacent the corner, and the material resistant to breakage extends around the corner between the first and second locations.

11. The digital X-ray detector of claim 1, comprising a plurality of the indicator.

12. A digital X-ray detector, comprising:
a detector array configured to generate image data based on incident X-ray radiation;
a housing in which the detector array is disposed, wherein the housing comprises a plurality of corners; and
a plurality of indicators, wherein each indicator of the plurality of indicators is disposed located at a respective corner of the plurality of corners, each indicator of the plurality of indicators comprises at least one light source, and the plurality of indicators together are configured to provide a single user perceptible signal indicating a single status of the digital X-ray detector.

13. The digital X-ray detector of claim 12, wherein the single status comprises whether the digital X-ray detector is ready for acquiring the image data.

14. The digital X-ray detector of claim 12, wherein each indicator of the plurality of indicators is configured to identify an active area of the digital X-ray detector for acquiring the image data.

15. The digital X-ray detector of claim 14, wherein each indicator of the plurality of indicators is configured to indicate the active area in a first direction and a second direction crosswise to the first direction.

16. The digital X-ray detector of claim 15, wherein each indicator of the plurality of indicators comprises a first light source that is configured to indicate the active area in the first direction and a second light source configured to indicate the active area in the second direction.

17. A method, comprising:
determining a single status of a digital X-ray detector; and
providing a user perceptible signal indicating the single status of the digital X-ray detector via an indicator disposed adjacent a corner of a housing of the digital X-ray detector, wherein the indicator comprises at least one light source.

18. The method of claim 17, wherein the indicator identifies an active area of the digital X-ray detector for acquiring image data by indicating the active area in a first direction and a second direction crosswise to the first direction.

19. The method of claim 17, wherein the single status comprises whether the digital X-ray detector is ready for acquiring image data.

20. The method of claim 17, comprising providing the user perceptible signal indicating the single status of the digital X-ray detector via a plurality of the indicator, wherein each indicator is disposed at a respective corner of the housing.
